(19) United States
(12) Patent Application Publication
(10) Pub. No.: US 2025/0152907 A1
(43) Pub. Date: May 15, 2025

(54) Multi-Sensory Training Protocol and Apparatus

(71) Applicant: EyeLux Integrations, LLC, Columbus, OH (US)

(72) Inventor: Steven J. Curtis, Columbus, OH (US)

(73) Assignee: EyeLux Integrations, LLC, Columbus, OH (US)

(21) Appl. No.: 18/506,189

(22) Filed: Nov. 10, 2023

(51) Int. Cl. A61M 21/00

(52) U.S. Cl. CPC A61M 21/00; A61M 2021/0022; A61M 2021/0027; A61M 2021/0044

(57) ABSTRACT

A protocol uses vision, vestibular, somatosensory, and auditory stimuli simultaneously. This presentation of multiple sensory stimuli to a participant provides opportunity to observe the participant's response to the stimuli. The protocol incorporates use of a motorized horizontal platform, a lighted visual display, a music-audio device, oculomotor activities, and a computerized controller. The controller is operable via a software application that provides various multi-sensory stimulation protocols. The program includes instructions executable by the software application to cause movement of the motorized horizontal platform, lighted visual display to emit a visual stimulus, to play the audio-music stimulus, and to provide the participant with oculomotor activities.

### CROSS-REFERENCE TO RELATED APPLICATIONS

### STATEMENT REGARDING FEDERALLY SPONSORED RESEARCH AND DEVELOPMENT

Not applicable.

### THE NAMES OF THE PARTIES TO A JOINT RESEARCH AGREEMENT

Not applicable.

### REFERENCE TO A “SEQUENCE LISTING,” A TABLE, OR A COMPUTER PROGRAM

Not applicable.

### STATEMENT REGARDING PRIOR DISCLOSURES BY THE INVENTOR OR A JOINT INVENTOR

Not applicable.

### BACKGROUND OF THE INVENTION

The present disclosure relates to delivering multi-sensory training and more particularly to a novel protocol therefor.

In various situations it is desirable to deliver multiple stimuli to a participant and to evaluate the participant's reaction to the stimuli. Some devices have been developed for this purpose. Examples of prior devices can be seen, for example, in U.S. Pat. Nos. 4,728,294, 6,651,279, 6,656,137, 9,675,776, 4,320,0768, 10,940,260, and others.

However, there still exists a need for advanced systems and methods for introducing multiple stimuli to a participant for evaluating a reaction of the participant to the multiple stimuli.

It is to such need that the present invention is addressed.

### BRIEF SUMMARY OF THE INVENTION

OMST™ and MST (trademarks of Eyelux Integrations, LLC) is a protocol using vision, vestibular, somatosensory, and auditory stimuli simultaneously. This presentation of multiple sensory stimuli to a participant provides opportunity to observe the participant's response to the stimuli. The protocol incorporates use of a motorized horizontal platform, a lighted visual display, a music-audio device, oculomotor activities, and a computerized controller. The controller is operable via a software application that provides various multi-sensory stimulation protocols. The program includes instructions executable by the software application to cause the lighted visual display to emit a visual stimulus, to play the audio-music stimulus, and to provide the participant with oculomotor activities. The motorized horizontal platform the participant lies upon is controlled separately by a digital timer. The system also may have the table controlled from the software application—one button starts all stimuli.

Various embodiments of the present invention provide systems and methods for introducing sensory input to a participant, in a controlled fashion. This includes introducing motion, sound, somatization, and visual senses in a carefully synchronized presentation capable of inducing a participant reaction without over stimulating the participant. The table moves at a defined rate; the audio device emits an audible stimulus; and the light emitting visual display emits a visual stimulus.

The methods include providing a movement table, providing a light emitting visual display, providing an audio device, and providing a controller. The controller is electrically connected to the movement table, the audio device, and the light emitting visual display. The methods further comprise delivering a command to the controller via a communication network. The command at least in part controls the stimuli delivered by at least one of the movement table, the light emitting display, and the audio device. Further, in some cases, the command is operable to preclude use of at least one of the movement table, the light emitting visual display, and the audio device. In other cases, the command is operable to cause a modulated musical sound to emanate from the audio device, while in yet other cases the command is operable to cause a series of uniform colors to display via the light emitting display and/or to control a rate at which the movement table moves.

The present invention provides a multi-sensory introduction of systems. The systems include a movement table that is operable to introduce a movement sense to a participant supine on the table, an auditory input device that is operable to introduce an audio sense to the participant, and a visual input device that is operable to introduce a visual sense to the participant. The systems further include a controller that is communicably coupled of the auditory device and the visual input device and the motion table. The movement table is a trochoidal motion table and the movement sense is produced in accordance with the table motion. In operation, such a trochoidal motion table can be utilized to maintain the head of the participant fixed in relation to the body of the participant.

In some cases, the auditory input device is further operable to receive a song and to segregate the song into at least one song segment, and wherein the audio sense introduced to the participant includes at least one song segment. In some cases, the song segment or segments are created by attenuating the sound level during portions of the song such that the sound output is segmented. This segmentation can include partially attenuating the output level of the sound such that it is still audible and/or attenuating portions of the song such that it becomes inaudible. Thus, a segment can be a portion of a song played at one output level and terminating when the output level is modified to play at a different output level. Based on the disclosure provided herein, one of ordinary skill in the art will recognize various song segments that can be created and used in accordance with embodiments of the present invention. In various cases, a song is received by the auditory input device and output such that the song is presented as a modulated musical sound which is then introduced to the participant. In some cases, the modulated musical sound is created by attenuating the output level of portions of the song relative to other portions of the song. The song is received by the controller.

The visual input device is operable to receive a visible spectrum light sequence command, and the visual sense includes one or more visible spectra light outputs presented in accordance with the light sequence command. The controller is further operable to prevent operation of at least one of the auditory device and the visual input device and the motion table in accordance with a operational command. An operational command preventing operation is received.

This summary provides only a general outline of some embodiments of the present invention. Many other objects, features, advantages, and other embodiments of the present invention will become more fully apparent from the following detailed description, the appended claims, and the accompanying drawings.

### BRIEF DESCRIPTION OF THE SEVERAL VIEWS OF THE DRAWINGS

For a fuller understanding of the nature and advantages of the present method and process, reference should be had to the following detailed description taken in connection with the accompanying drawings, in which:

FIG. 1 is an isometric view of the apparatus including a movement table, providing stimuli including a light emitting visual display, an audio device, and a controller;

FIG. 2 discloses the method steps implemented by the software stored in the controller;

FIG. 3 is a continuation of the method steps of FIG. 3.

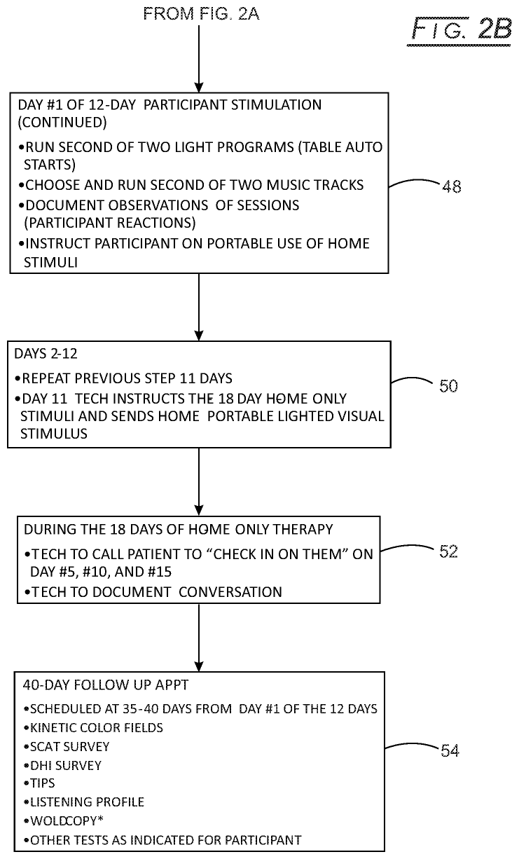

The drawings will be described in greater detail below.

### DETAILED DESCRIPTION OF THE INVENTION

Various embodiments of the present invention provide systems and methods for introducing sensory input to a participant, and in particular one or more embodiments of the present invention provide systems and methods for introducing sensory input in a controlled fashion to a plurality of human sensory receptors and for controlling such introduction remotely. In some cases, such embodiments include introducing motion, sound, somatosensory and visual senses in a carefully synchronized presentation capable of inducing a participant reaction without over stimulating the participant.

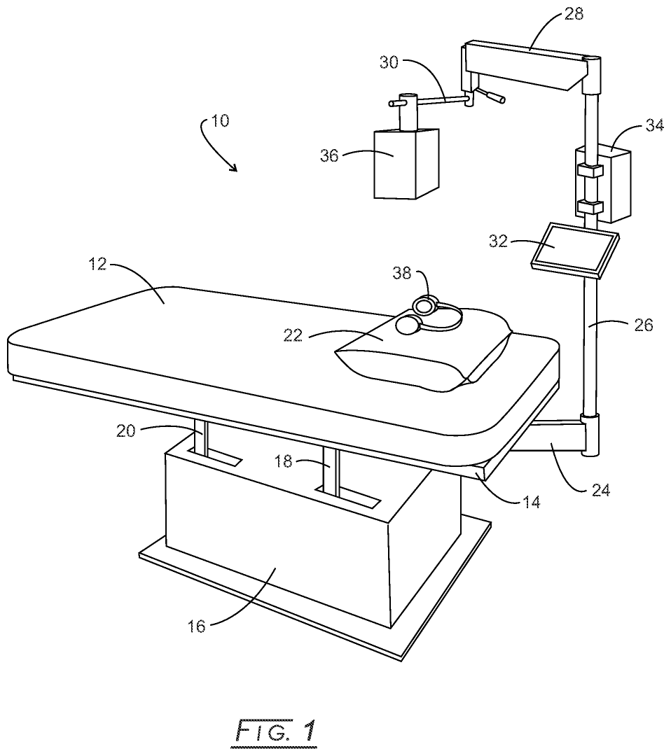

Turning to FIG. 1, a multi-sensory introduction system 10 in accordance with some embodiments of the present invention is depicted. System 10 includes a visual input device, 32, a trochoidal movement table, 12, an auditory input device, 38, and a controller, 34. Further, controller 34 is communicably coupled to a auditory input device, 38, movement table 12, and visual input device 36. As used herein, the term “communicably coupled” is used in its broadest sense to include any connection or method whereby information can be transmitted between devices. Thus, for example, devices can be communicably coupled via a wire, an optical coupling, an RF or other wireless coupling, and/or the like. As used herein, the term “electrically coupled” is a subset of communicably coupled and implies the use of a wire extending between coupled devices. Also, as used herein, the term “communication network” is used in its broadest sense to mean any network whereby information can be passed. Thus, for example, controller 34 may be connected to the Internet, a PSTN, a virtual private network, a peer-to-peer network, a WAN, a LAN, and/or any combination thereof or the like. While the exemplary system of FIG. 1 shows wireless coupling between controller 34 and the various stimulating devices, one of ordinary skill in the art will recognize that various media can be used to provide the various communicable couplings.

Movement table 12 can be any table capable of moving a participant disposed thereon using, for example, a pillow, 22, for comfort. In some embodiments of the present invention, movement table 12 is a trochoidal movement table capable of providing a smooth movement pattern. In some instances, the movement pattern is a round or smooth movement pattern that avoids sudden changes in direction. In one particular instance, the movement pattern is a circular movement pattern in either a clockwise rotation, or a counterclockwise rotation. The rate of movement can be adjusted such that a participant disposed on movement table 12 does not perceive that they are moving, but rather perceives that stationary objects around the participant are moving. In one particular case, the movement table is adjusted such that it moves along a circular path with a diameter of between four and eleven inches and at a rate of between seven and fourteen revolutions per minute. In one particular case, the table rotates along a circular path with a diameter of seven inches and at a rate of ten to eleven revolutions per minute and is capable of inducing vestibular stimulation in an anterior/posterior orientation (e.g., similar to doing somersaults one after another) and a lateral orientation (e.g., similar to rolling over and over like a log). In some cases, this motion can arouse the reticular activating system of a participant's brain. The tabletop may be rotated ninety degrees so that a participant is exposed to anterior/posterior movement in one session and lateral movement in the next session, while in supine position in all instances.

It will be observed that movement table 12 is carried by two pair of upstanding legs, 18 and 20 (two of which do not show in the drawings) that extend into a base, 16, that houses a motor, actuator, or other motive device (not shown) for effecting the noted modes of movement of movement table 12.

Visual input device 36 is carried by a laterally extending arm, 30, that is moveably connected to a generally laterally extending arm, 28, which in turn is carried by a generally extending rotatable vertical pole, 26, that in turn is affixed to base 16 by a generally laterally extending moveable arm 24. Pole 26 also carries controller 34 and visual input device, 32.

Visual input device 36 can be any device capable of displaying a controlled visible spectrum light pattern and creating a visual sense for a participant disposed on movement table 12. In one particular embodiment, visual input device 36 includes a circular opening or aperture through which light is passed. In such an embodiment, a series of colored lights of the visible spectrum can be passed though such circular opening. This series of colored lights can be, for example, ruby, red, yellow-green, blue-green, blue, violet, pink, or magenta with each color displaying in series for a defined period of time. In some cases, this series of colors can be presented automatically, as dictated by a sensory program executed, for example, by controller 34. Examples of such control can be provided via a sensory program which is discussed below in more detail. In some cases, visual input device 36 is stationary relative to movement table 12, such that a participant disposed on movement table 12 can be moved through space relative to stationary visual input device 36.

In one particular embodiment of the present invention, visual input device 36 includes an aperture of between one and four inches in diameter through which light is displayed to a participant. In one particular case, the aperture has a diameter of nominally about two inches. In some cases, the aperture is disposed between fifteen and twenty-two inches above a participant's eyes when the participant is disposed on movement table 12. In one particular instance, the aperture is disposed between seventeen and twenty inches above a participant's eyes when the participant is disposed on movement table 12. Providing the visual stimulation to a participant may, for example, trigger sympathetic and parasympathetic systems and changes in the firing pattern of the hypothalamus of the participant when visual light on the red end of the spectrum is presented, and a sedating effect on the blue end of the spectrum. Further, providing the visual stimulation may exercise the extrinsic eye muscles and enhance the ability of the eyes to track and team together. Further, providing the visual and movement stimuli together may aid the participant in adapting to the combination of stimuli and strengthen the vestibular ocular reflex. Also, the end of the spectrum can trigger the extrinsic eye muscles to shape the eyeball as if looking in the near point and the red end of the spectrum triggers the muscles as if looking in a distance. Such changes may exercise a participant's accommodation skills.

Auditory input device 38, such as headphones, can be any device capable of outputting an audible signal to a participant. In some embodiments of the present invention, auditory input device 140 is capable of receiving a song which can be any series of audible notes and modulating the song. In some cases, the modulation is done by a software program executed by a microprocessor. The software program reduces the output level or volume that is presented to the participant at varying intervals during the song. Thus, for example, three seconds of the song may be played at a first volume level, a subsequent second portion of the song can be played at a reduced volume level, and a subsequent two seconds of the song can be played at the first volume level. In some cases, the reduced volume level is not audible by the participant, while in other cases it is audible. This modulation creates song segments. Further, the modulation can be applied with a number of different volume levels, or only with two volume levels. Yet further, the modulation can be applied at random time orders and/or random volume levels or can be presented in a non-random predictable sequence or some combination of random and non-random. In one particular case, elements of a song between 20 and 1000 Hz are modulated in one way and elements of the song between 1000 and 20000 Hz are modulated in another way. The separately modulated song portions can then be recombined to create a single modulated musical song. This modulated musical song can then be presented to a participant to create an audio sense.

Controller 34 can be any microprocessor-based device, optionally capable of communicating with remote server and issuing commands to one or more of movement table 12, visual input device 36, and auditory input device 38. In one particular embodiment, controller 34 is a personal computer (PC) capable of executing one or more software programs that result in control signals. For example, one software program can be a sensory program that directs the introduction of various sensory input to a participant via one or more of movement table 12, visual input device 36, and auditory input device 38. Alternatively, or in addition, software can be included to control the operational status of one or more of movement table 12, visual input device 36, and auditory input device 38. Thus, for example, it may be determined by remote server that software license fees have not been paid and that operation of the system no longer exists. In such a case, a remote server may communicate a command that is executed by controller 34 to stop operation of the effected system portions.

The remote server can be any microprocessor-based system capable of communicating with controller 34 via a wireless or wired communication network. Thus, as just some examples, the remote server can be a PC, a network server, or the like. Based on the disclosure provided herein, one of ordinary skill in the art will recognize that remote server can be a single unified device such as a PC or can be a number of microprocessor-based devices, each performing one or more functions attributed to remote server. Similarly, the remote server can be a single unified device, or a distributed group of devices with each performing different functions.

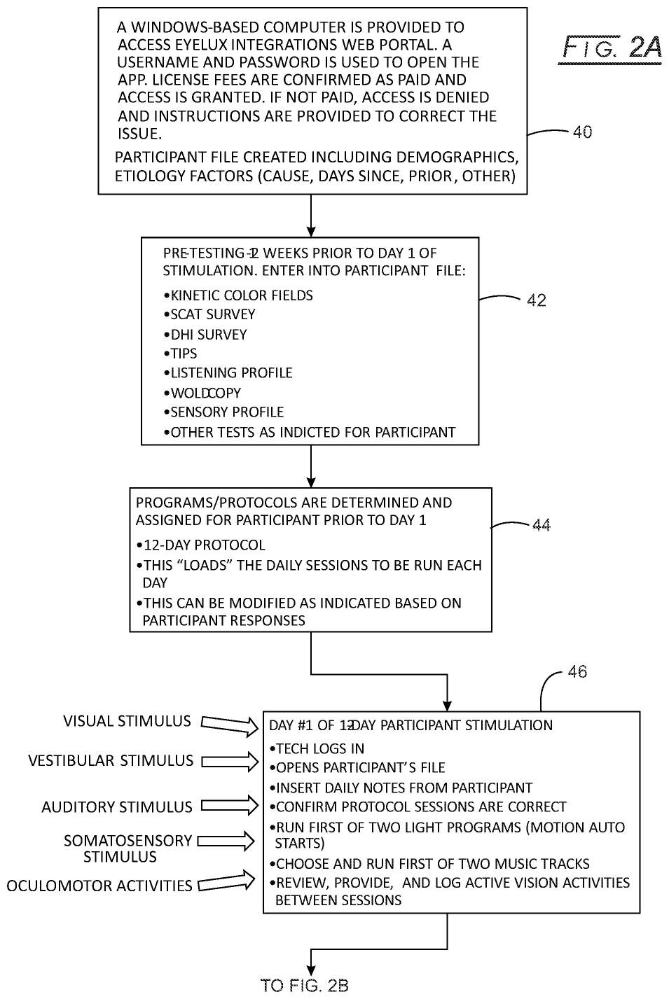

Turning to FIG. 2A, a block diagram of a multi-sensory program or software capable of implementing the disclosed embodiments of the present invention is illustrated. Such software can be housed within controller 34 or in a remote computer system in communication with controller 34. For present purposes it is immaterial whether the disclosed stimuli patterns are remotely controlled or not.

For a revenue model that focuses on remote control of controller 34, the software commences with block 40 wherein controller 34 commences its operations by accessing a web portal using the typical username and password mode familiar to all Internet users. Providing that the user logging is in good stead of license fees being current, controller 34 now is in communication with the software controlling operation of multi-sensory introduction system 10. If the user is not current in license fee payment, instructions are provided for the user to bring its account up to date. Whether remote or local, the software includes entry of data for each participant being treated including, for example, demographics, etiology factors, and the like.

In step 42, the participant is pre-tested 1-2 weeks prior to stimulation treatment and data is entered into the file (computer memory) of the participant, such as, for example, Kinetic color fields, SCAT survey, DHI survey, TIPS, listening profile results, Wold Copy, Sensory profile, and other test appropriate for the participant.

In step 44, the software continues to entry into the computer the programs/protocols determined and assigned to the participant for Day 1 and the subsequent 12-day protocol. Each day, the software effects the programs/protocols stored in memory for that particular participant. Of course, daily results may result in modifications being made to those items stored in memory.

In step 46, the software continues by commencing with the Day 1 entry. A variety of stimuli singularly and/or in various combinations available for the participant include, inter alia, visual stimulus, vestibular stimulus, auditory stimulus, somatosensory stimulus, and oculomotor activities. The technician logs into the system and opens the participant's file. The technician can enter daily notes of the participant and confirms that the protocol session is correct. The software is engaged to run, for example, the first of two light programs (motion of the table commences automatically). The first of two music tracks is chosen and run. Finally, the technician reviews, provides, and logs active vision activities between sessions.

The software continues in FIG. 2B by running the second of the two light programs with the table automatically engaged per that light program protocol. The second of the two music tracks is engaged also. The technician records observations of the sessions (e.g., participants reactions). The technician instructs the participant on portable use of home stimuli appropriate for that participant between sessions.

In step 50, the foregoing activities are repeated for days 2 through 12. After session 11, the technician instructs the participant on the 18-day home only stimuli and sends the participant home with a portable lighted visual stimulus.

In step 52, the technician monitors the home therapy by calling the patient on day numbers 5, 10, and 15. Those conversations are documented in the patient's file.

In final step 54, a follow-up appointment for between 35 to 60 days post Day 1 is scheduled. At that appointment, the pretests are repeated, viz., such as, for example, Kinetic color fields, SCAT survey, DHI survey, TIPS, listening profile, Wold Copy, Sensory profile, and other test appropriate for the participant.

Having communication between controller 34 and a remote computer system allows for the trained professional to be located remote from the sensory input system to dictate or interact in the operation of the system. This can be particularly advantageous where selecting a sensory program for use in relation to the system involves considerable training. Based on the disclosure provided herein, one of ordinary skill in the art will recognize a number of other uses and/or advantages that may be had through use of a remote server in relation to a sensory input system.

In one particular application of a sensory input system in accordance with an embodiment of the present invention, the system is used to provide multiple sensory stimuli to a participant. In some cases, the participant can be a person that has a somewhat limited ability to combine multiple stimuli into a single experience. In some cases, such as when a person may suffer from what is commonly referred to as autism. Evaluating and/or treating autism is more fully described in the following two articles, the entirety of which are incorporated herein by reference for all purposes: “Cortical Activation and Synchronization During Sentence Comprehension in High-Functioning Autism: Evidence of Underconnectivity”, Just et. al., BRAIN a Journal of Neurology Vol. 127, No. 8; “Merging Sensory Signals in the Brain: The Development of Multisensory Integration in the Superior Colliculus”, Stein et al., Development of the New Cognitive Neurosciences, MIT Press, Cambridge, Mass., 2^{nd} ed. (2000).

The sensory input system provides a tool for the controlled introduction of multiple sensory stimuli in such a way that the participant is not overwhelmed. This allows the participant to, in a sense, practice incorporating multiple stimuli, and in some cases, gain a greater ability to cope with complex situations. This operates to provide an educational experience for the participant through the integrated and controlled use of multiple sensory stimuli including, but not limited to, visual colored light, modulated musical sound, and vestibular stimulation (e.g., motion). Stated another way, such a sensory system can help a person organize their sensations. In some cases, the auditory input device provides modulated musical sound. The recurring attenuation in the modulated musical sound makes it difficult for the participant to become secure in the musical tune; thus, continually bringing the participant present by helping the participant to shift attention from point to point. Further, in some cases, a higher volume is introduced to one of the participant's ears relative to the other. In some cases, a higher volume is introduced to the right ear than to the left which may help to improve a participant's right ear dominance.

Further, in some cases, listening characteristics of a participant (i.e., one example of participant characteristics) are measured or otherwise obtained. This can include measuring the amplitude and/or frequency of an audible output that can be detected by a participant. This information can be assembled into a listening profile indicating a number of characteristics of the participant's listening abilities. In some cases, a screening audiometer can be used to develop the listening profile. It may be that the listening profile is more representative of a participant's ability to maintain attention and process auditory stimuli than the participant's sensitivity to auditory stimuli. Thus, in some cases, the listening profile may be an indication of the any unevenness between ears in a participant's hearing thresholds. Based on the disclosure provided herein, one of ordinary skill in the art will recognize a number of possible approaches for identifying a participant's listening characteristics and/or assembling a listening profile.

In various cases, the visual characteristics (i.e., one example of participant characteristics) of a participant also are measured or otherwise obtained. In so doing, a visual field chart can be completed upon exposing a participant to various colored (i.e., frequencies of visual light) 1-3 mm targets presented at various angles relative to the participant. For evaluation, a participant is instructed to centralize their vision at a central point. At this time colored targets (white, red, blue, green) stimulating color cones of the participant's eye are slowly moved toward the central point from the periphery. When the participant indicates that they can see the color, a color mark is made in each of eight pie-shaped segments on a chart. This colored chart is in essence a perception map of the back of the participant's eye (i.e., the periphery, and the area around the blind spot). These colored marks provide an indication of a neurotypical amount of photocurrent traveling along the optic nerve or whether a restrictive amount is traveling along the optic nerve. A constricted amount, as indicated by a limited field of view, may indicate an impediment associated with the visual system of the participant.

In another method for delivering sensory stimuli to a participant, the participant's characteristics are identified, such as, for example, visual and auditory anomalies exhibited by the participant that may affect the way a participant perceives stimuli. As just some examples, when a participant is rotated through space on the movement table and is presented with various stimuli, such characteristics can include: a tendency by a participant to turn their head to one side possibly indicating that the vision abilities of the participant are not well established; the participant perceives that the light appears divided into two lights that may indicate a convergence problem; the participant is unable to direct a laser pointer at a stationary object may indicate a level of disorientation; the participant reports that they are not lying flat, but feels like they are “lying on a hill” may indicate a vestibular problem that affects posture and/or sense of posture; the participant exhibits a high degree of fear possibly indicating a weak vestibular, visual and auditory integration; the participant is restless and agitated, but becomes calm when an alphabet or number sequence is counted may indicate a degree of hyperlexia and where concrete sensations of movement have been overwhelming; the participant does not see the light moving may indicate that the vestibular system is not influencing the visual system normally possibly inhibiting the post-rotatory nystagmus reflex; the participant has difficulty following the light when rotated horizontally and crossing the midline can indicate a horizontal eye muscle problem; the participant has difficulty following the light when rotated vertically and crossing the eye level which may be an indication of a vertical eye muscle problem (e.g., Brown's syndrome, where there is a limitation of upward gaze); the participant is seen to develop an oral fixation, such as beginning to suck their thumb or to chew on something may indicate that the sensory program has “switched on” the oral/motor system; the participant becomes receptive to the auditory stimulation, this appears to make listening come alive, by stimulating the stirrup bone and the trapezius with gated reflexes to the muscles involved with speech; and the participant becomes itchy and scratchy around the head and rubs eyes, this is the start of breaking down the tactile defensiveness, and normalizes hypo-reactions to pain (this can be manifest in a change from not wanting to be touched to wanting to be touched). Various characteristics can be identified before the participant is exposed to any stimuli (pre-sensory), or after the participant is already exposed to some stimuli (post-sensory). One or more of these characteristics are recorded in the file of the participant and the characteristics are used to identify a sensory program particularly suited to the characteristics.

As just one example, when the visual field shows anomaly of difficulty moving eye or eyes upward to denote visual stimulus, the movement table can be programmed to move the participant anterial-posterial (head to toe) across the horizon line when the extrinsic eye muscle is inhibited from being raised to gaze. As some other examples, a left ear threshold above the right ear threshold can be relied upon to remotely cause an increase (or weighting) of the right ear volume to establish correct right ear dominance, or where an inhibition in initiation and execution of speech in response to auditory presentations a binaural rather than monaural presentation of music may be indicated. As yet another example, when a visual field is constricted to less than five degrees, a thirty minute light session rather than a shorter light session may be selected by a trained professional located on site or remote from the participant.

As an example, the light input portion can be tailored to select between a number of color programs that can be displayed via visual input device 36. In one embodiment, tens to hundreds of color programs are available providing different color sequences displayed across 20 minutes, 25 minutes, 30 minutes, or a 20 minute interval followed by ten minutes of darkness. As another example, the movement input portion can command table 12 to operate at a particular rate (adjustment of rotation speed and reverse direction is available with a separate switches) during the time when the light program is being presented, and to stop movement when the light program completes.

As just one example, it may be determined if the visual field of the participant has expanded indicating that more photocurrent is travelling along the optic nerve. These newly observed characteristics can then be communicated to the remote server or to controller 34.

As just one example, a twelve year old participant with a constricted visual field and uneven listening profile with a divergent peak (i.e., both ears moving in different sensitivity directions) at 8 KHz may be exposed on a movement table to a thirty minute light series and monaural acoustic program filtered at 12 KHz. Where the child becomes emotionally overwhelmed and is experiencing headaches, a switch to binaural modulation with a reduction in volume and change in light series may be indicated. With these changes, it may be found that the participant adapts more easily and successfully to the sensory program.

While the apparatus, system, and method have been described with reference to various embodiments, those skilled in the art will understand that various changes may be made and equivalents may be substituted for elements thereof without departing from the scope and essence of the disclosure. In addition, many modifications may be made to adapt a particular situation or material in accordance with the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the American engineering system and all amounts and percentages are by weight, unless otherwise expressly indicated. Also, all citations referred herein are expressly incorporated herein by reference.

1. A system for providing multi-sensory stimuli to a participant comprising:
(a) a trochoidal movement table for supporting, moving (vestibular), and somatosensory input to a participant;
(b) a light emitting visual display located for viewing by a patient supported on the trochoidal movement table;
(c) an audio device located for hearing of audio signals emitted from the audio device by a participant supported on the trochoidal movement table;
(d) a processor controller in control communication with the trochoidal movement table, the light emitting visual display, and the audio device;
wherein the controller is operable to receive and store a sensory software program from a server, wherein the sensory software program includes instructions executable by the processor to cause movement of the trochoidal movement table in one of at least two selectable directions, several speed options, cause light emissions from the light emitting visual display, and cause sound to emit from the audio device.

2. The system of claim 1, wherein the server is remotely located.

3. The system of claim 2, wherein the controller additionally has a second software program stored which second software program is operable in response to a characteristic of the participant.

4. The system of claim 3, wherein the characteristic of the participant is evaluated upon interaction of the participant to stimuli produced in relation to the first sensory program.

5. The system of claim 4, wherein the controller has stored a sensory program received from the remove server, wherein the sensory program includes instructions executable by the processor to cause movement of the trochoidal movement table at a defined rate of movement, to cause the audio device to emit an audible stimulus, and the light emitting visual display to emit a visual stimulus.

6. A method for delivering a multi-sensory stimuli to a participant comprising the steps of:
(a) providing a trochoidal movement (vestibular) and somatosensory table for supporting a participant;
(b) providing a light emitting visual display viewable by a patient supported on the trochoidal movement table;
(c) providing an audio device located for emitting audio signals to a participant supported on the trochoidal movement table;
(d) providing a controller in control communication with the trochoidal movement table, the light emitting visual display, and the audio device, the controller including a processor and a stored sensory software program;
(e) delivering a command from the stored sensory software program to cause one or more of movement of the trochoidal movement and somatosensory table in one of at least two selectable directions, causes light emissions from the light emitting visual display, or causes sound to emit from the audio device.

7. The method of claim 6, further including a step of providing a remove server in control communication with the controller.

8. The method of claim 7, further including a step of providing the controller with a stored second software program operable in response to a characteristic of the participant.

9. The method of claim 8, wherein further including a step of evaluating the characteristic of the participant based upon interaction of the participant to stimuli produced in relation to the first sensory program.

10. The method of claim 9, further including a step of the remote server providing a sensory program to the controller, wherein the sensory program includes instructions executable by the processor to cause movement of the trochoidal movement table at a defined rate of movement, to cause the audio device to emit an audible stimulus, and the light emitting visual display to emit a visual stimulus.
